(19) United States
(12) Patent Application Publication
(10) Pub. No.: US 2023/0235581 A1
(43) Pub. Date: Jul. 27, 2023

(54) MEASUREMENT SYSTEM, APPARATUS, METHOD AND PROGRAM

(71) Applicant: NIPPON TELEGRAPH AND TELEPHONE CORPORATION, Tokyo (JP)

(72) Inventors: Rie SAKAI, Musashino-shi, Tokyo (JP); Hiroshi YOSHIDA, Musashino-shi, Tokyo (JP); Hiroshi OIWA, Musashino-shi, Tokyo (JP); Tomoko SHIBATA, Musashino-shi, Tokyo (JP)

(73) Assignee: NIPPON TELEGRAPH AND TELEPHONE CORPORATION, Tokyo (JP)

(21) Appl. No.: 18/010,159

(22) PCT Filed: Jun. 15, 2020
(86) PCT No.: PCT/JP2020/023390

§ 371 Date: Dec. 13, 2022

(51) Int. Cl. E04G 5/00; G01M 1/12; E04G 5/08

(52) U.S. Cl. CPC E04G 5/001; E04G 5/08; G01M 1/122; E06C 7/18

(57) ABSTRACT

A measurement system according to the present embodiment includes a work tool, a sensor unit, an acquisition unit, a change unit, and a calculation unit. The work tool has a tread board with a variable width. The sensor unit is provided in the work tool. The acquisition unit acquires, from the sensor unit, time-series data on gravity center sway of a worker, in a state where the worker is standing on the tread board. The change unit reduces the width of the tread board in response to a trigger. The calculation unit calculates, from the time-series data, an evaluation value for the gravity center sway of the worker, for each width of the tread board.

### TECHNICAL FIELD

The present invention relates to a measurement system, a measurement apparatus, a measurement method, and a program.

### BACKGROUND ART

There has been a demand for a technique for identifying a dangerous action of a worker working at a high altitude, such as swaying and falling. For example, from a value acquired by a sensor while a worker is working on a planer sheet provided with a plurality of the sensors in advance, an ability of the worker to maintain balance can be analyzed.

### CITATION LIST

### Non Patent Literature

NPL 1: Anima Co., Ltd., “BALANCE CODER BW-6000”, [online], [Search on Jun. 1, 2020], internet <URL: https://anima.jp/products/bw6000/>

### SUMMARY OF THE INVENTION

### Technical Problem

Unfortunately, the analysis is not practical in terms of sensory and psychological aspects of the worker, because the work by the worker on the sheet provided with the sensors is different from a work on a scaffold or tread board usually used. On top of that, the widths of the scaffold and the tread board used by the worker vary among products. In this context, the analysis on the action of the worker on a single sheet only enables the acquisition of one type of analysis data corresponding to the size of the sheet. A plurality of sheets with different sizes may be prepared, but this method is costly and requires the measurement to be repeatedly performed. Thus, such a method is cumbersome for the worker and is insufficient.

The present invention has been made in view of the above circumstances, and an object of the present invention is to provide a measurement system, a measurement apparatus, a measurement method, and a program with which a change in sway of a worker can be efficiently measured.

### Means for Solving the Problem

To achieve the above described object, a measurement system according to an aspect of the present invention includes a work tool, a sensor unit, an acquisition unit, a change unit, and a calculation unit. The work tool has a tread board with a variable width. The sensor unit is provided in the work tool. The acquisition unit acquires, from the sensor unit, time-series data on gravity center sway of a worker, in a state where the worker is standing on the tread board. The change unit reduces the width of the tread board in response to a trigger. The calculation unit calculates, from the time-series data, an evaluation value for the gravity center sway of the worker, for each width of the tread board.

A measurement apparatus according to an aspect of the present invention includes an acquisition unit, a change unit, and a calculation unit. The acquisition unit acquires, from a sensor unit provided in a work tool having a tread board with a variable width, time-series data on gravity center sway of a worker, in a state where the worker is standing on the tread board. The change unit reduces the width of the tread board in response to a trigger. The calculation unit calculates, from the time-series data, an evaluation value for the gravity center sway of the worker, for each width of the tread board.

### Effects of the Invention

Thus, with the present invention, a change in sway of a worker can be efficiently measured.

### BRIEF DESCRIPTION OF DRAWINGS

FIG. 1 is a block diagram illustrating a measurement system including a measurement apparatus according to the present embodiment.

FIG. 2 is a diagram illustrating an outer appearance of an example of a work tool of the present embodiment.

FIG. 3 is a diagram illustrating an example of a reduction mechanism for a tread board according to the present embodiment.

FIG. 4 is a flowchart illustrating an operation of the measurement apparatus according to the present embodiment.

FIG. 5 is a diagram illustrating an example of a method for determining gravity center deviation performed by a determination unit.

FIG. 6 is a diagram illustrating an example of a measurement report according to the present embodiment.

FIG. 7 is a diagram illustrating an example of a graph related to an evaluation value according to the present embodiment.

FIG. 8 is a diagram illustrating an example of work information table stored in a work information database according to the present embodiment.

### DESCRIPTION OF EMBODIMENTS

Hereinafter, a measurement system, a measurement apparatus, a measurement method, and a program according to an embodiment of the present disclosure will be described in detail with reference to the drawings. Note that parts with the same numbers applied thereto will not be repeatedly described in the following embodiments on the assumption that the parts perform similar operations.

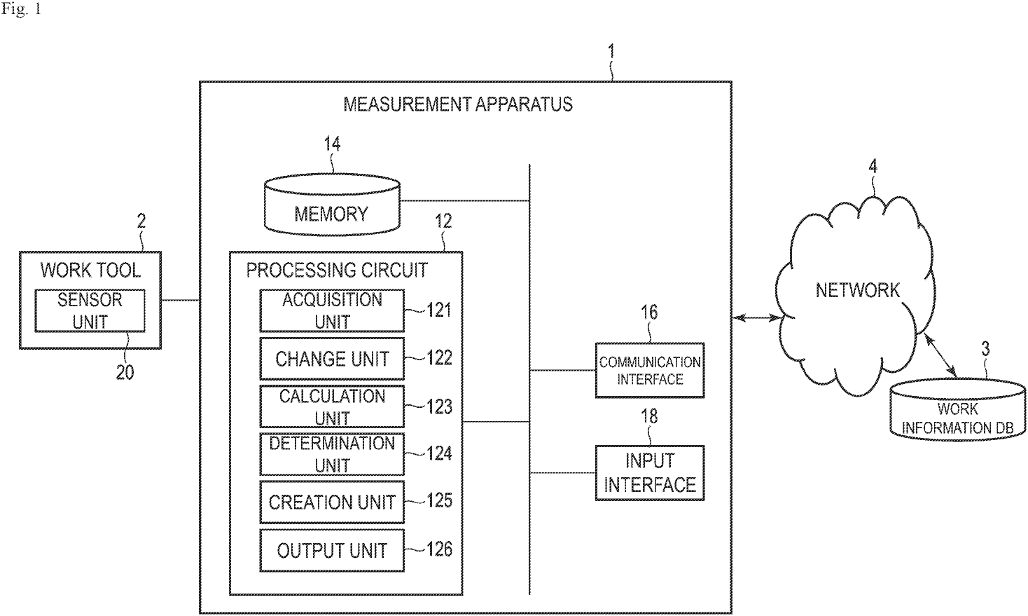

A measurement system including a measurement apparatus according to the present embodiment will be described with reference to FIG. 1. The measurement system according to the present embodiment includes a measurement apparatus 1, a work tool 2, and a work information database 3.

The measurement apparatus 1 and the work tool 2 are connected to each other in a wireless or wired manner, to be capable of exchanging data therebetween. The measurement apparatus 1 and the work information database 3 are connected to each other via a network 5 in a wireless or wired manner. Although one measurement apparatus 1 is illustrated in the example illustrated in FIG. 1, a plurality of the measurement apparatuses 1 may be connected to one work information database 3. Furthermore, one measurement apparatus 1 may be connected to a plurality of the work tools 2.

The work tool 2 has a tread board with a variable width. The present embodiment is described with the work tool 2 assumed to be a stand in a form of a stepladder including the tread board with a variable width, and leg parts supporting the tread board, but this should not be construed in a limiting sense. The work tool 2 may be any tool used for measurement operation performed by a worker on the tool. Possible examples of such a tool include a tripod, a workbench, and a scaffolding platform, as well as a tool in a form of a tread board alone such as a sensor board. Each of the leg parts of the work tool 2 is provided with a sensor unit 20. The sensor unit 20 acquires weight-related sensor values that change depending on the movement of the center of gravity of the worker. The sensor used as the sensor unit 20 is, for example, a strain sensor capable of measuring a pressure value.

The measurement apparatus 1 includes a processing circuit 12, a memory 14, a communication interface 16, and an input interface 18. The processing circuit 12 includes an acquisition unit 121, a change unit 122, a calculation unit 123, a determination unit 124, a creation unit 125, and an output unit 126. The sensor unit 20 is connected to other components included in the measurement apparatus 1 in a wired or wireless manner. The processing circuit 12, the memory 14, the communication interface 16, and the input interface 18 are connected to one another, for example, via a bus.

The acquisition unit 121 acquires time-series data related to the gravity center sway of the worker, in a state where the worker is standing on the tread board, from each of the sensor units 20.

The change unit 122 reduces the width of the tread board of the work tool 2 in response to a trigger. The change unit 122 stops the reduction of the width of the tread board in response to a stop instruction from the determination unit 124.

The calculation unit 123 calculates, from the time-series data, an evaluation value for the gravity center sway of the worker, for each width of the tread board. Examples of the evaluation value include a gravity center sway area and a maximum value of variation range of the gravity center trajectory in individual axial direction.

The determination unit 124 determines whether the trigger has occurred. The determination unit 124 transmits the stop instruction to the change unit 122, when the weight related to the tread board decreases to or below a threshold value, or when the gravity center position of the worker projected on a plane of the tread board moves out from the region of the tread board (may be referred to as gravity center deviation in the present embodiment) for a predetermined number of times. When the evaluation value is equal to or greater than a threshold value, the determination unit 124 may determine that the state of the worker working is unstable. Specific examples of the case where the state of the worker is unstable include a state where the worker is unbalanced and swaying, a state in which the worker is likely to fall from the high-place work tool, and the like.

The creation unit 125 creates a measurement report including identification information on the worker, the width of the tread board, and the corresponding evaluation value of the worker.

The output unit 126 outputs the measurement report. The measurement report may be transmitted to the work information database 3. When a display (not illustrated) is connected to the measurement apparatus 1, the report may be displayed on the display viewable by the worker him or herself, other workers, or a manager.

The processing circuit 12 is composed of a processor such as a central processing unit (CPU) or an integrated circuit such as a field programmable gate array (FPGA) or an application specific integrated circuit (ASIC). Each of the above-described processing units (acquisition unit 121, change unit 122, calculation unit 123, determination unit 124, creation unit 125, and output unit 126) may be implemented as one function of the processor or the integrated circuit by the processor or the integrated circuit executing a processing program, or may be implemented by a plurality of ASICs executing functions of the respective processing units.

The memory 14 stores data such as the sensor value, the evaluation value, the identification information of the worker, and the measurement report. For example, the memory 14 may be a generally used storage medium such as a hard disk drive (HDD), a solid state drive (SSD), or a flash memory. In a situation where the measurement apparatus 1 is capable of transmitting and receiving data to and from the work information database 3 via the network 5, the measurement apparatus 1 may transmit data (sensor value, evaluation value, identification information, and the like) to the work information database 3 every time the state detection device 1 acquires and generates the data, and thus the memory 14 does not need to store the previous data. In this case, the memory 14 may be a temporary storage medium with a volatile memory such as a cache memory.

The communication interface 16 is an interface for a data communication between the sensor unit 20, the work information database 3, and the measurement apparatus 1. As the communication interface 16, a commonly used communication interface, such as a wired LAN or a wireless LAN may be used, and thus descriptions thereof will be omitted.

The input interface 18 is, for example, a mouse, a keyboard, a switch, a button, or a touch panel display and receives an input from a user of the measurement apparatus 1.

The work information database 3 stores the identification information, the evaluation value, the measurement report, and the like for each worker. An average measurement report and an average evaluation value may be further stored for each age. Although, it is assumed that the work information database 3 is provided in, for example, a cloud server and communicates with a plurality of measurement apparatuses 1, the work information management database 3 may be stored in a dedicated server.

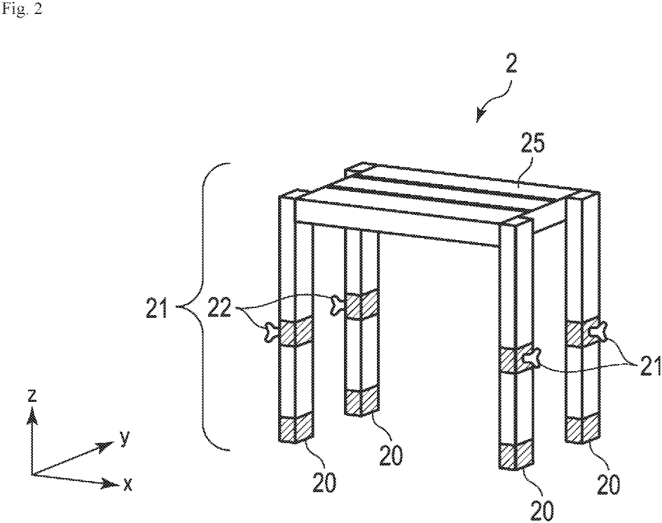

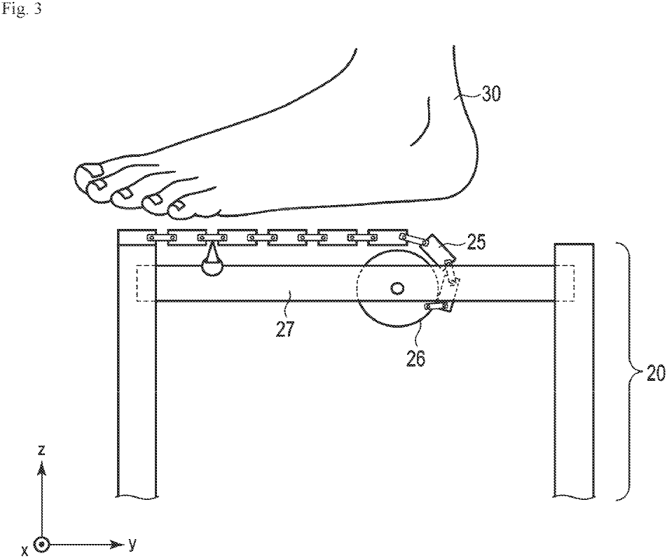

Next, an example of the work tool 2 according to the present embodiment will be described with reference to FIG. 2 and FIG. 3. FIG. 2 is a diagram illustrating an outer appearance of the work tool 2. A tread board 25 of the work tool 2 is formed of a member capable of supporting the load of the worker so that the worker can stand on and move off from the tread board 25. The tread board 25 is formed to have a reducible width. Each of legs 21 of the work tool includes a telescopic mechanism 22 with which the length of the leg 21 can be adjusted. For the telescopic mechanism 22, a mechanism may be used in which a portion accommodated in the leg 21 extends when a lock portion is opened and the length of the leg 21 is fixed when the lock portion is closed. The lock portion is assumed to be of a screw type in the example illustrated in FIG. 2, but may employ any generally used lock mechanism such as a clip type or a fitting type. The telescopic mechanism 22 of each leg 21 is independently adjustable.

In the present embodiment, the sensor unit 20 is assumed to be attached to a ground contact-side tip of the leg 21. A slip resistant grip made of rubber or the like is typically provided on the tip of the leg 21. Thus, the sensor unit 20 may be disposed between the slip resistant grip and the tip of the leg 21, the sensor unit 20 may be embedded in the slip resistant grip itself, or a member that has a slip resistant function and includes the sensor unit 20 from the top of the slip resistant grip may be provided at the tip of the leg 21. Note that the sensor unit 20 may be embedded in the leg 21.

It is assumed that the sensor unit 20 acquires the pressure value as the sensor value, but the sensor may acquire, as the sensor value, other types of information such as the sensing time, the altitude, the temperature, and the magnetic field. In the example of FIG. 2, with the four sensor units 20 disposed on the respective legs 21, it is possible to acquire, as the sensor value, the pressure when the worker stands on the work tool 2 from each of the sensor units 20, and the load related to the sensor unit 20 can be calculated. When the worker stands on the work tool 2, the pressure on the sensor unit 20 changes. Thus, it is possible to detect that the worker has stood on the work tool 2. Furthermore, by continuously acquiring the sensor values from the positions of the four sensor units 20 at a predetermined interval, it is possible to acquire time-series data of the sensor values. Based on the time-series data, a change in the center of gravity of the worker can be calculated. Note that the number of the sensor units 20 arranged is not limited to four, as long as a change in the center of gravity of the worker can be calculated, and it suffices if the sensor units 20 are provided at at least three portions. When the work tool has a shape of a plate composed of the tread board 25 alone, the sensor units 20 may be arranged at four corners or three of four corners of the ground side surface of a frame supporting the tread board 25 for example. All things considered, the sensor units 20 may be arranged at any position as long as the load applied to the tread board 25 can be measured.

The sensor unit 20 includes a tag recognition unit that senses an ID recognition tag held by the worker. The ID recognition tag includes a worker ID for uniquely identifying the worker. The sensor unit 20 recognizes the ID recognition tag of the worker attempting to stand on the work tool 2 for a work and acquires the worker ID of the worker standing on the work tool 2 and the time at which the worker stands on the work tool 2. For example, the recognition of the ID tag by the sensor unit 20 may have a configuration, such as a scheme using near field communication (NFC), capable of recognition in a manner that the worker approaches the ID recognition tag to the sensor unit 20 or brings the ID recognition tag into contact with the sensor unit 20 or have a configuration, such as a scheme using RFID for example, enabling the sensor unit 20 to recognize the ID recognition tag which is within a predetermined range from the sensor unit 20.

Instead of identifying the worker ID with the ID recognition tag, the worker ID of the worker standing on the work tool 2 may be identified in a manner that the worker inputs the own worker ID to the input interface 18 of the measurement apparatus 1 and then performs a work.

Next, an example of a reduction mechanism for the tread board 25 of the work tool 2 will be described with reference to FIG. 3. As illustrated in FIG. 3, the tread board 25 is formed to be in a form of a belt conveyor, and can have the width reduced when a roller 26 rotates to wind the tread board 25 along a guide 27. As a result, a region of the tread board 25 on which a foot 30 of the worker can be placed can be reduced.

The roller 26 is driven by, for example, a motor (not illustrated), and is controlled to rotate and stop, with the motor driven based on an instruction signal from the measurement apparatus 1. The rotation and stop may be manually implemented by a worker or a manager operating the measurement apparatus using a switch separately attached. The reduction mechanism for the tread board 25 is not limited to the structure comprising the roller 26 and the tread board 25 in a form of a belt conveyor, and any mechanism may be used as long as the width of the tread board 25 can be reduced. Possible configurations include one in which the tread board 25 is formed by a plurality of plates which are removable for reducing the region of the tread board 25 and the like. In the example illustrated in FIG. 3, the width of the tread board 25 is reduced without changing the distance between the legs 21 of the work tool 2. Alternatively, the distance between the tips of the legs 21 on the tread board side may be reduced in accordance with the reduction of the width of the tread board 25. Thus, the state where the legs 21 are connected to the corners of the tread board may be constantly maintained.

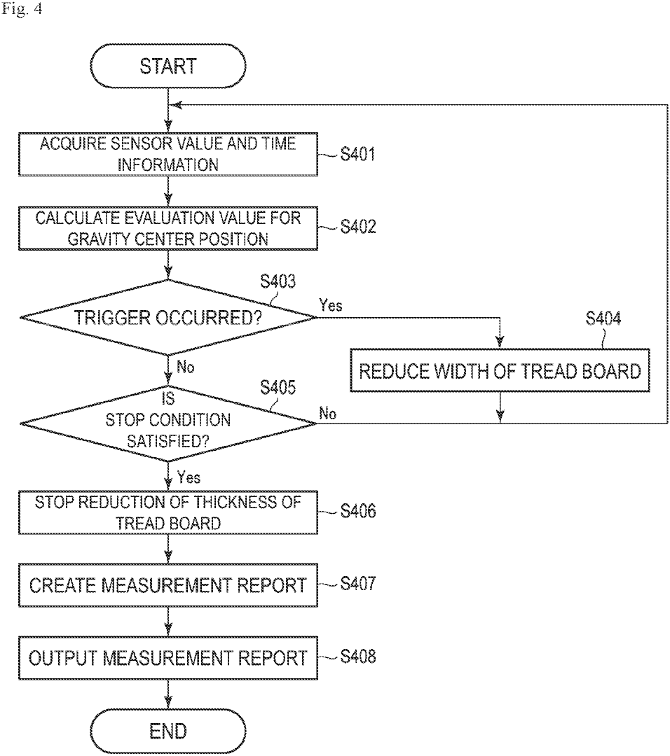

Next, an operation of the measurement apparatus 1 according to the present embodiment will be described with reference to the flowchart in FIG. 4.

When the measurement starts, in step S401, the acquisition unit 121 acquires, from the sensor unit 20, the sensor value, the worker ID, and the time at which the worker has stood on the work tool 2. The sensor value is time-series data sampled at a predetermined interval. The time at which the worker has stood on the work tool 2 is defined as measurement start time. The measurement may be triggered to be started as follows. Specifically, when the worker stands on the tread board 25, the load applied to the sensor units 20 exceeds 0, and thus the measurement apparatus 1 may determine the measurement start time, with the measurement determined to be started in response to application of the load that is equal to or greater than the threshold value on the sensor units 20. Alternatively, the measurement may start in response to the worker touching a “start button” displayed on the display, or pressing a button of an input device such as a keyboard. In this case, the time at which the start button is pressed is determined as the measurement start time. When the worker inputs the own worker ID to the input interface 18 of the measurement apparatus 1, and then performs a work, the time at which the worker ID is input may be determined as the measurement start time.

In step S402, the calculation unit 123 calculates the center of gravity of the worker from the sensor value and calculates the evaluation value for the gravity center sway. Regarding the center of gravity of the worker, if the sensor values at the legs of the stepladder are equal to one another, it can be calculated that the center of gravity of the worker is at the center (for example, center of a work region of the worker, which is defined by the four legs of the stepladder) of a planar region defined by the arrangement of the four sensors. Thus, by comparing the changes of the respective sensor values that are time-series data, it is possible to calculate the position of the center of gravity of the worker in the planar region. When the evaluation value is the gravity center sway area, the calculation unit 123 may calculate the gravity center trajectory each time a new sensor value is acquired, to calculate the gravity center sway area in real time. As a method for calculating the gravity center sway area, a general method of calculating an area may be used such as, for example, one using an area based on the contour of the trajectory of the center of gravity as the gravity center sway area. Thus, description thereof will be omitted. When the evaluation value is the maximum value of the variation range of the gravity center trajectory in axial directions, the maximum value and the minimum value of coordinates of the calculated center of gravity in a vertical direction and a horizontal direction may be calculated, and a difference between the maximum value and the minimum value may be obtained, and then the variation range may be calculated.

In step S403, the determination unit 124 determines whether the trigger has occurred. It may be determined that the trigger has occurred when a predetermined period of time elapses with the current width of the tread board 25, or when the worker or the manager managing the measurement presses a button for changing the width of the tread board 25. In accordance with a determination that the trigger has occurred, the processing proceeds to step S404, and in accordance with a determination that the trigger is has not occurred, the processing proceeds to step S405.

In step S404, the change unit 122 reduces the width of the tread board 25 in response to the occurrence of the trigger. Specifically, the change unit 122 transmits the instruction signal for reducing the width of the tread board 25 to the work tool 2, and the roller of the work tool 2 rotates to reduce the width of the tread board 25 by a predetermined width. The predetermined width to be reduced is assumed to be a predetermined distance such as 1 cm. Note that the reduced width may be changed as appropriate depending on the condition of the worker. Specifically, the reduced width may be initially large and gradually get smaller. Then, the processing returns to step S401, and the same processing is repeated.

In step S405, the determination unit 124 determines whether a condition for stopping the reduction of the width of the tread board 25 is satisfied. It is determined that the stop condition is satisfied when the weight on the tread board 25 becomes equal to or lighter than a threshold value, that is, when the sensor values from the sensor units 20 decrease to or below the threshold value, because this indicates that the worker is expected to have fallen from or moved off from the work tool 2. The stop condition It is also determined that the stop condition is satisfied when the gravity center deviation occurs for a predetermined times, because this indicates that the worker is swaying and thus is expected to be in an unbalanced and unstable state. In accordance with a determination that the stop condition is satisfied, the processing proceeds to step S406. In accordance with a determination that the stop condition is not satisfied, the processing returns to step S401, and the same processing is repeated. Thus, with the processing in step S401 to step S405 repeated, the width of the tread board 25 gradually decreases as long the worker is not in an unstable state.

In step S406, the change unit 122 stops the reduction of the width of the tread board 25. A description will be given regarding the reduction of the width of the tread board 25 of the work tool 2 by the change unit 122. The instruction signal for the stop is transmitted, and on the work tool 2 side, the roller is controlled to be stopped without rotating in response to the trigger. In step S407, the creation unit 125 creates the measurement report including each width of the tread board 25 and the corresponding evaluation value, until the reduction of the width of the tread board 25 stops. In step S408, the output unit 126 outputs the measurement report to the outside, that is, to the display, the work information database 3 connected to the measurement apparatus 1, or the like.

Note that the order in which step S403 and step S405 are processed is not important, and step S403 and step S405 for determining whether the condition is satisfied may be processed by the determination unit 124 in parallel.

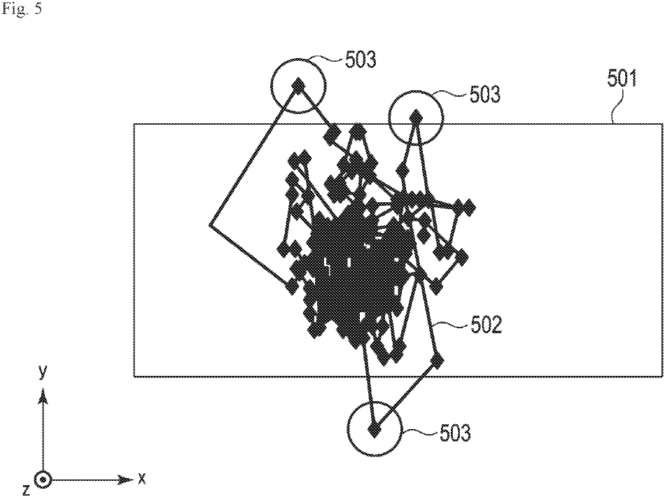

Next, an example of a method for determining the gravity center deviation performed by the determination unit 124 will be described with reference to FIG. 5. A region 501 illustrated in FIG. 5 is the region of the tread board 25 of the work tool 2 as viewed in the orthogonal direction (z-axis direction). A trajectory 502 is time-series data indicating the trajectory of the gravity center position of the worker, and represents the gravity center position projected on the plane of the tread board 25. As illustrated in FIG. 5, when the state of the worker becomes unstable, the gravity center position is located outside of the region 501 of the tread board 25, resulting in a gravity center deviation point 503. The determination unit 124 may count the number of such gravity center deviation points 503, to determine that the stop condition is satisfied when the gravity center deviation occurs for a predetermined number of times.

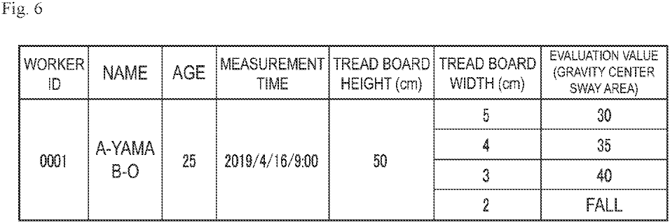

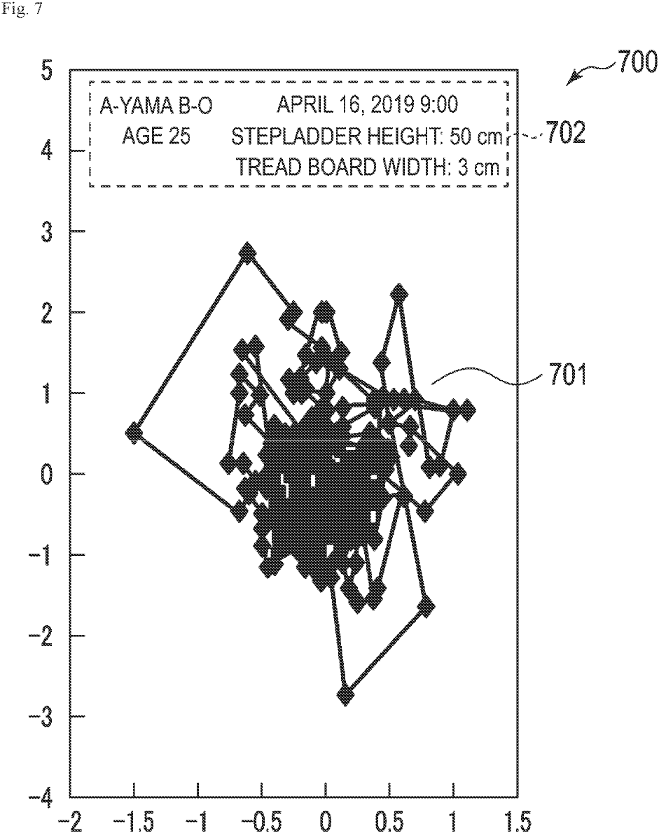

Next, an example of the measurement report according to the present embodiment will be described with reference to FIG. 6 and FIG. 7. FIG. 6 illustrates an example in which, information in which the worker name, the measurement time, the height of the tread board 25, the width of the tread board 25, and the evaluation value are associated with each other is output as the measurement report. In this example, the information is displayed in a table format. As the width of the tread board 25 and the evaluation value, each reduced width of the tread board 25 and the evaluation value obtained with the width are associated with each other. In the example illustrated in FIG. 6, the gravity center sway area, obtained each time the width of the tread board 25 is reduced by 1 cm, is used for the evaluation value. It can be seen that the worker has fallen and the measurement has ended when the width of the tread board 25 is 2 cm. Note that information can be included as the measurement report.

A change in the gravity center sway area in accordance with a change in the width of the tread board can be recognized at a glance by seeing the measurement report, and thus the width that has resulted in an unstable state of the worker can be easily recognized.

FIG. 7 is a graph 700 related to the evaluation value. Specifically, a trajectory 701 related to the gravity center sway is displayed, and on the upper side of the graph 700, work data 702 is displayed that includes the name “A-YAMA B-O”, the age “25”, the measurement start time “Apr. 16, 2019 9:00”, the tread board height “50 cm”, and the tread board width “3 cm”. The graph 700 illustrated in FIG. 7 may be the graph 700 displayed that corresponds to the evaluation value the region of which has been clicked by the worker or the manager for example. Alternatively, the graph 700 corresponding to all evaluation values for the width in the measurement report may be displayed at once.

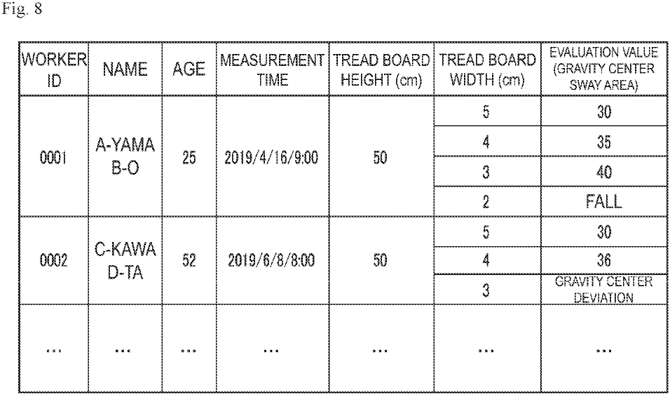

Next, FIG. 8 illustrates an example of a work information table that includes the measurement report, and is stored in the work information database 3. For a plurality of workers, the individual measurement reports, illustrated in FIG. 6, are compiled in a work information table 800 illustrated in FIG. 8.

In the example illustrated in FIG. 8, the work information table 800 stores a measurement report for a person with the worker ID “0001”, the name “A-YAMA B-O”, and the age “25” and a measurement report for a person with the worker ID “0002”, the name “C-KAWA D-TA”, and the age “52”. For example, the creation unit 125 can extract the measurement report with the name “A-YAMA B-O”, by searching the work information table 800 using the worker ID “0001” as the key.

As the measurement report stored in the work information table 800, only the past measurement reports may be remained, or only the latest measurement report may be stored. By referring to the work information table 800, the creation unit 125 may create statistical data that can be calculated from the information in the work information table 800, such as the average value of the measurement report for each age, for each tread board height, or the like. When displaying the measurement report to a worker, the output unit 126 may also display the statistical data created for each age and/or each tread board height to the worker.

According to the present embodiment described above, a sensor is attached to a work tool having a tread board with a variable width, time-series data on gravity center sway is acquired while reducing the width of the tread board, and an evaluation value such as gravity center sway area corresponding to the width of the tread board is calculated to create a measurement report. With this configuration, the worker can calculate the evaluation value such as the gravity center sway area with various widths of the tread board by a single measurement and determine the unstableness such as swaying and risk of falling of the worker in accordance with the width of the tread board, without going through cumbersome measurements performed by repeatedly standing on a plurality of work tools having tread boards with different widths. Furthermore, with the measurement report output, the worker on the tread board or a person around him or her can be notified of the balance status of the worker corresponding to the width of the tread board, using the evaluation value and the like. As a result, it is possible to efficiently measure a change in swaying of a worker, while securing the safety of the worker.

The instructions indicated in the processing procedures described in the embodiment described above can be executed by a computer based on a software program. The program can be recorded on a recording medium, or can be provided via a network.

In short, the present invention is not limited to the above-described embodiments as it is and can be embodied by modifying the constituent components within the scope not departing from the gist of the disclosure when implemented. Furthermore, various inventions can be formed by appropriately combining a plurality of constituent components disclosed in the above embodiments. For example, several constituent elements may be omitted from all the constituent elements illustrated in the embodiments. Furthermore, components in different embodiments may be appropriately combined with each other.

Reference Signs List

| | |
|---|---|
| 1 | Measurement apparatus |
| 2 | Work tool |
| 3 | Work information database |
| 5 | Network |
| 12 | Processing circuit |
| 14 | Memory |
| 16 | Communication interface |
| 18 | Input interface |
| 20 | Sensor unit |
| 21 | Leg |
| 22 | Telescopic mechanism |
| 25 | Tread board |
| 26 | Roller |
| 27 | Guide |
| 30 | Foot |
| 121 | Acquisition unit |
| 122 | Change unit |
| 123 | Calculation unit |
| 124 | Determination unit |
| 125 | Creation unit |
| 126 | Output unit |
| 501 | Region |
| 502, 701 | Trajectory |
| 503 | Point |
| 700 | Graph |
| 702 | Work data |
| 800 | Work information table |

1. A measurement system comprising:
a work tool having a tread board with a variable width;
a sensor unit provided in the work tool;
an acquisition unit configured to acquire, in a state where a worker is standing on the tread board, time-series data on gravity center sway of the worker from the sensor unit;
a change unit configured to reduce the width of the tread board in response to a trigger; and
a calculation unit configured to calculate, for each width of the tread board, an evaluation value related to the gravity center sway of the worker from the time-series data.

2. The measurement system according to claim 1, wherein when a weight on the tread board decreases to or below a threshold value, or when a gravity center position of the worker, projected on a plane of the tread board, deviates from a region of the tread board for a predetermined number of times, the change unit stops the reduction of the width of the tread board.

3. The measurement system according to claim 1 further comprising a creation unit configured to create a measurement report in which the width of the tread board and the evaluation value of the worker corresponding to the width of the tread board are associated with each other.

4. The measurement system according to claim 1, wherein the calculation unit calculates, as the evaluation value, a value including a gravity center sway area of the worker or a maximum value of a variation range in individual axial direction of a gravity center trajectory of the worker.

5. A measurement apparatus comprising:
a processor; and
a storage medium having computer program instructions stored thereon, when executed by the processor, perform to:
acquire from a sensor unit provided in a work tool having a tread board with a variable width, time-series data on gravity center sway of a worker in a state where the worker is standing on the tread board;
reduce the width of the tread board in response to a trigger; and
calculate, for each width of the tread board, an evaluation value related to the gravity center sway of the worker from the time-series data.

6. The measurement apparatus according to claim 5, wherein when the worker moves off from the tread board, or when a gravity center position of the worker, projected on a plane of the tread board, deviates from a region of the tread board for a predetermined number of times, the change unit stops the reduction of the width of the tread board.

7. A measurement method comprising:
acquiring from a sensor unit provided in a work tool having a tread board with a variable width, time-series data on gravity center sway of a worker in a state where the worker is standing on the tread board;
reducing the width of the tread board in response to a trigger; and
calculating, for each width of the tread board, an evaluation value related to the gravity center sway of the worker from the time-series data.

8. A non-transitory computer-readable medium having computer-executable instructions that, upon execution of the instructions by a processor of a computer, cause the computer to function as the measurement apparatus according to claim 5.
